(19) United States
(12) Patent Application Publication
(10) Pub. No.: US 2019/0161724 A1
(43) Pub. Date: May 30, 2019

(54) SUSPENSION CELL CULTURE MONITORING APPARATUS

(71) Applicant: VIEWORKS CO., LTD., Anyang-Si (KR)

(72) Inventors: Youjung Kang, Anyang-Si (KR); Hooshik Kim, Anyang-Si (KR)

(73) Assignee: VIEWORKS CO., LTD., Anyang-Si (KR)

(21) Appl. No.: 16/196,291

(22) Filed: Nov. 20, 2018

(30) Foreign Application Priority Data
Nov. 20, 2017 (KR) 10-2017-0154555

(51) Int. Cl. C12M 1/34; C12M 1/12; C12M 1/00

(52) U.S. Cl. CPC C12M 41/46; C12M 31/06; C12M 25/00

(57) ABSTRACT

The present invention relates to a suspension cell culture monitoring apparatus including a chamber unit, which is a portion submerged in an incubator in which cells are cultured, that has at least a portion opened by an opening so that a culture medium and suspension cells are allowed to pass therethrough, a lens unit configured to zoom in or zoom out the suspension cells moving through the opening of the chamber unit, which are visible through an observation area of the chamber unit, by adjusting a focus, and a sensor unit configured to acquire an image in which the suspension cells visible through the lens unit are present.

### CROSS-REFERENCE TO RELATED APPLICATION

This application claims priority to and the benefit of Korean Patent Application No. 10-2017-0154555, filed on Nov. 20, 2017, the disclosure of which is incorporated herein by reference in its entirety.

### BACKGROUND

### 1. Field of the Invention

The present invention relates to a suspension cell culture monitoring apparatus, and more particularly, to a suspension cell culture monitoring apparatus capable of automatically monitoring a state of suspension cell culture and sending an analysis result thereof to a user.

### 2. Discussion of Related Art

In recent years, with the growth of the biopharmaceutical market, a need for the mass culture of cells has been increased. In particular, the biopharmaceutical manufacturing industry using free-floating cells has grown rapidly.

However, at present, due to the absence of a system capable of automatically monitoring suspension cell culture, it is common to culture cells without detailed information on a state of the cell culture or to culture cells by checking the cells by performing manual monitoring several times every day.

Accordingly, conventionally, in order to observe a state of cells during the cell culture, it is necessary to collect some cells from a culture apparatus, but such a process has problems in that it increases the probability of contamination of the cell culture and consumes a large workforce. Furthermore, if contamination occurs during the process of culturing a large number of cells, there is a problem in that a great economic and time loss is caused.

When, for the purpose of preventing the occurrence of such problems, cells are cultured for a certain amount of time without observing a state of the cells, the concentration of the cells may excessively increase or the morphology of the cells may be changed during the culturing process, and a change in the quality of biopharmaceuticals may be caused. This causes another problem in that the change in quality may be a fatal flaw in securing the quality in a pharmaceutical process.

Accordingly, there has been a demand for an apparatus which prevents the above-listed problems and is capable of automatically monitoring suspension cell culture.

The related art of the present invention has been disclosed in Korean Patent Publication No. 10-2016-0092553 (Date of Publication; Jan. 27, 2015, Title of Invention: Method and apparatus for real-time monitoring and feedback control of cell and tissue culture).

### SUMMARY OF THE INVENTION

According to an aspect of the present invention, the present invention has been devised to solve the above-listed problems, and it is an object of the present invention to provide a suspension cell culture monitoring apparatus capable of automatically monitoring a state of suspension cell culture and providing an analysis result thereof to a user.

A suspension cell culture monitoring apparatus according to an aspect of the present invention includes a chamber unit, which is a portion submerged in an incubator in which cells are cultured, configured to be opened at least partially by an opening so that a culture medium and suspension cells are allowed to pass therethrough, a lens unit configured to zoom in or zoom out the suspension cells moving through the opening of the chamber unit, which are visible through an observation area of the chamber unit, by adjusting a focus, and a sensor unit configured to acquire an image in which the suspension cells visible through the lens unit are present.

The observation area configured to allow observation of the suspension cells may be formed at one side of the chamber unit, and an illumination unit configured to illuminate the observation area may be coupled and integrally formed with the chamber unit.

The illumination unit may be formed to illuminate the observation area from an opposite side of the observation area by using an illumination cable.

The illumination unit may be formed to illuminate the observation area by sharing a lens barrel with the lens unit.

The suspension cell culture monitoring apparatus may be formed such that an outer portion thereof, excluding the opening of the chamber unit, is partially or entirely covered by a cover.

The cover may be formed of a waterproof material and sterilized.

A focusing operation, a zooming operation, and an iris operation of the lens unit may be controlled.

The suspension cell culture monitoring apparatus may further include a calculation unit configured to analyze the acquired image, wherein the calculation unit processes the image acquired by the sensor unit, analyzes a state of cell culture to extract statistical data, and outputs an alarm when the statistical data deviates from a set appropriate range, and the statistical data and the alarm may also be output through at least one of a server, a user computer, and a portable wireless terminal which is connected to the calculation unit via a communication network.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is an exemplary view illustrating a schematic configuration of a suspension cell culture monitoring apparatus according to an embodiment of the present invention;

FIG. 2 is a schematic diagram of the suspension cell culture monitoring apparatus according to an embodiment of the present invention illustrated in FIG. 1;

FIGS. 3A and 3B are schematic diagrams for describing an image capturing method using the suspension cell culture monitoring apparatus according to an embodiment of the present invention illustrated in FIG. 1;

FIGS. 4A and 4B are schematic diagrams for describing a method of mounting the suspension cell culture monitoring apparatus according to an embodiment of the present invention illustrated in FIG. 1 on an incubator;

FIGS. 5A and 5B are exemplary views illustrating a schematic configuration of a monitoring computer connected to the suspension cell culture monitoring apparatus according to an embodiment of the present invention; and

FIGS. 6A-6C are exemplary views for describing a monitoring operation of the monitoring computer illustrated in FIGS. 5A and 5B in more detail.

### DETAILED DESCRIPTION OF EXEMPLARY EMBODIMENTS

Hereinafter, an embodiment of a suspension cell culture monitoring apparatus according to the present invention will be described with reference to the accompanying drawings.

In this process, the thickness of lines, size of elements, or the like illustrated in the drawings may have been exaggerated for clarity and convenience of description. The terms which will be mentioned below are those defined in consideration of functions in the present invention, and the terms may vary according to intentions or practices of a user or an operator. Therefore, such terms should be defined on the basis of content throughout the present specification.

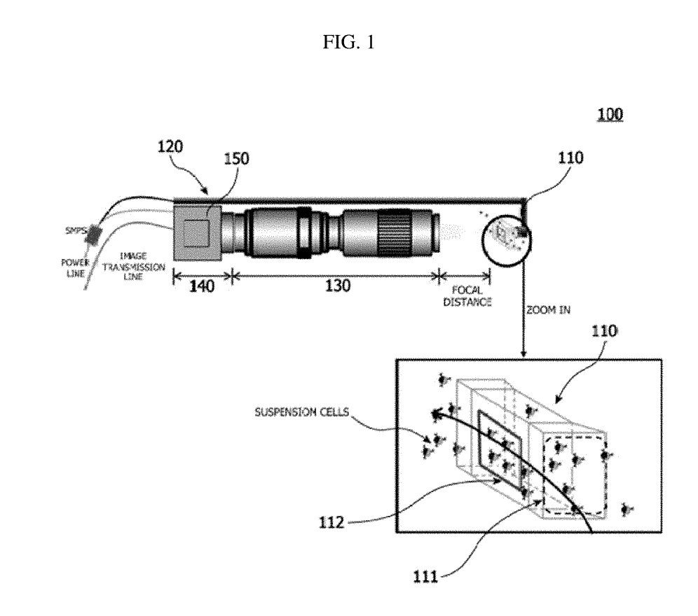

FIG. 1 is an exemplary viewer illustrating a schematic configuration of a suspension cell culture monitoring apparatus according to an embodiment of the present invention.

As illustrated in FIG. 1, a suspension cell culture monitoring apparatus 100 according to the present embodiment may include a chamber unit 110, an illumination unit 120, a lens unit 130, and a sensor unit 140 and may be integrally formed.

The chamber unit 110, which is a portion submerged in an incubator in which cells are cultured, has a structure in which it is opened by openings 111 formed at both ends so that a culture medium and suspension cells may pass through the chamber unit 110.

The chamber unit 110 has a structure in which suspension cells may flow in and out of the chamber unit 110 while an influence due to the flow of the culture medium is minimized

For example, the chamber unit 110 may have a structure in which the both ends are opened or a structure in which the both ends are partially opened.

Accordingly, the suspension cells which are cultured inside the incubator may flow and pass freely through the chamber unit 110.

An observation area 112 through which the suspension cells may be observed is formed at one side of the chamber unit 110 (in the present embodiment, the observation area 112 may be formed as a transparent window to allow the observation), and the illumination unit 120 is coupled (mounted) at an opposite side of the observation area 112. Alternatively, the illumination unit 120 may be formed to share an internal passage of the lens unit 130 outside the incubator and transmit light toward the observation area 112.

The observation area 112 allows an image of the suspension cells, which flow and pass through the chamber unit 110, to be captured using the lens unit 130 and the sensor unit 140.

The illumination unit 120 may be implemented to transmit light, which is emitted from outside the incubator, toward one side of the chamber unit 110 (that is, the opposite side of the observation area) by using an illumination cable (e.g., an optical cable), or may be implemented to transmit light toward the observation area 112 through the internal passage (or a lens barrel) of the lens unit 130, that is, to illuminate the observation area 112 in a direction identical to a gaze direction of the user.

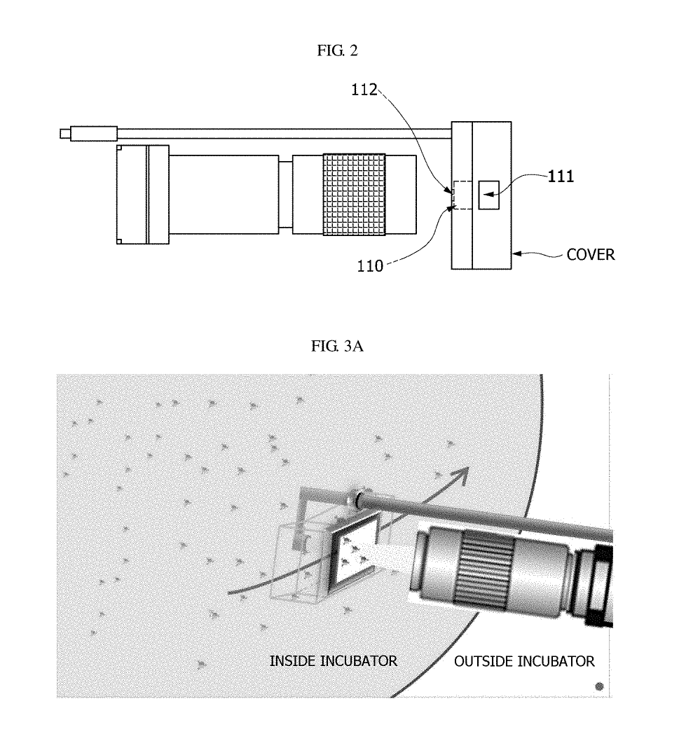

In addition, the chamber unit 110 and the illumination unit 120 are formed such that outer portions thereof, excluding the openings 111 formed at the both ends of the chamber unit 110, are entirely covered by an integral cover (see FIG. 2).

Accordingly, the culture medium in the incubator is prevented from being attached to the chamber unit 110 or the illumination unit 120 (that is, the illumination cable) so that contamination is prevented.

The cover which covers the chamber unit 110 and the illumination unit 120 prevents the culture medium from flowing into the apparatus 100 even when a portion (portion including the openings of the chamber unit) is submerged in the incubator. Accordingly, contamination of the apparatus 100 and the incubator is prevented.

FIG. 2 is a schematic diagram of the suspension cell culture monitoring apparatus according to an embodiment of the present invention illustrated in FIG. 1. FIG. 2 shows that the chamber unit 110 and the illumination unit 120 which are submerged in the incubator are not exposed to the culture medium due to removal of a portion of the cover.

The lens unit 130 may adjust a focus so that the suspension cells which are visible through the observation area 112 may be zoomed in/zoomed out or captured clearly.

The sensor unit 140 captures an image of the view visible through the lens unit 130 (that is, an image in which the suspension cells are present).

Accordingly, the sensor unit 140 includes an image sensor.

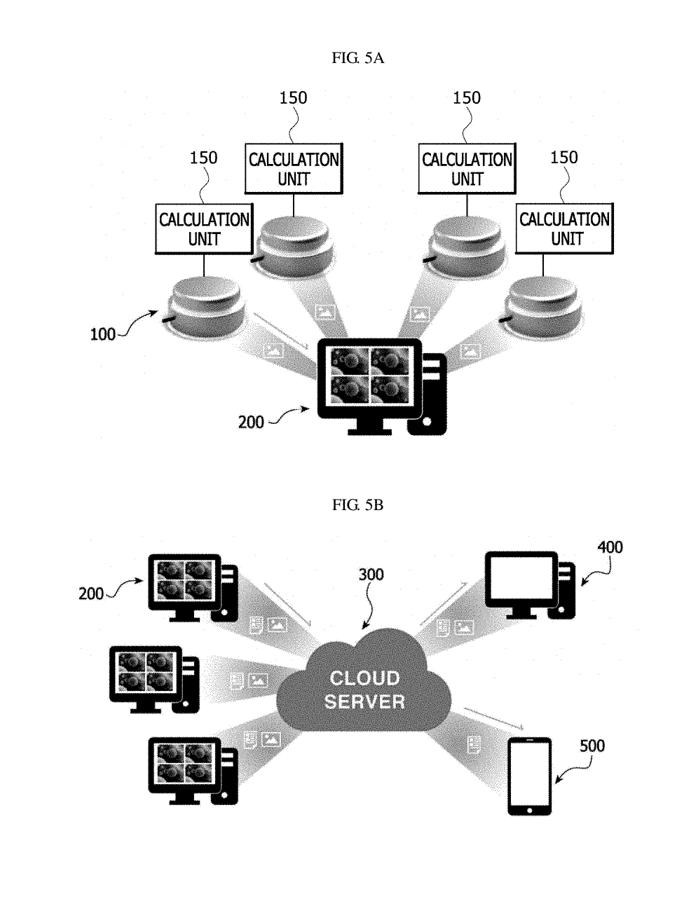

The image captured through the sensor unit 140 is sent to the outside (that is, a monitoring computer 200 (see FIGS. 5A and 5B)).

In addition, the sensor unit 140 may also be implemented to include a calculation unit 150.

That is, the sensor unit 140 may perform image processing (or analysis) as well as image acquisition. Therefore, acquisition and analysis of an image may be performed at once even when a separate monitoring computer is not included.

The lens unit 130 may be manually controlled or remotely controlled from the outside (that is, the monitoring computer 200 (see FIGS. 5A and 5B)).

For example, focusing, zooming, and an iris of the lens unit 130 may be controlled remotely (electrically).

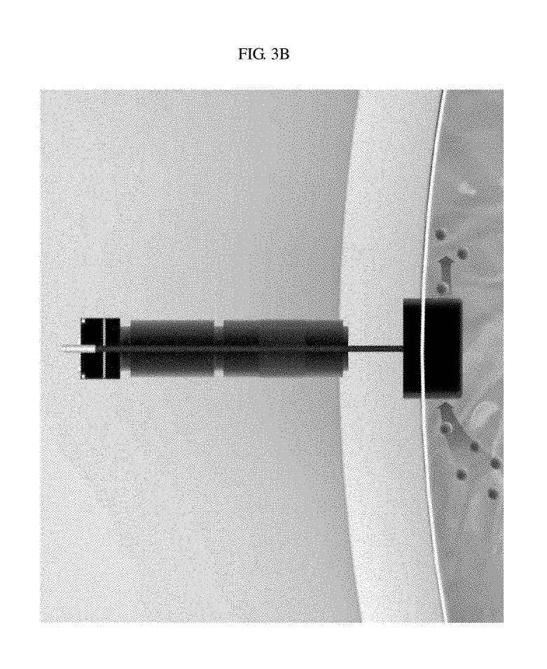

FIGS. 3A and 3B are schematic diagrams for describing an image capturing method using the suspension cell culture monitoring apparatus according to an embodiment of the present invention illustrated in FIG. 1. As illustrated FIG. 3A and FIG. 3B, light is irradiated toward the observation area 112 of the chamber unit 110 by using an illumination mounted at a rear surface of the chamber unit 110 while only a portion of the chamber unit 110 (portion including the openings of the chamber unit) is caused to be in the incubator, and an image of the suspension cells visible through the observation area 112 is captured through the lens unit 130 which is spaced a predetermined distance (focal distance) from the observation area 112.

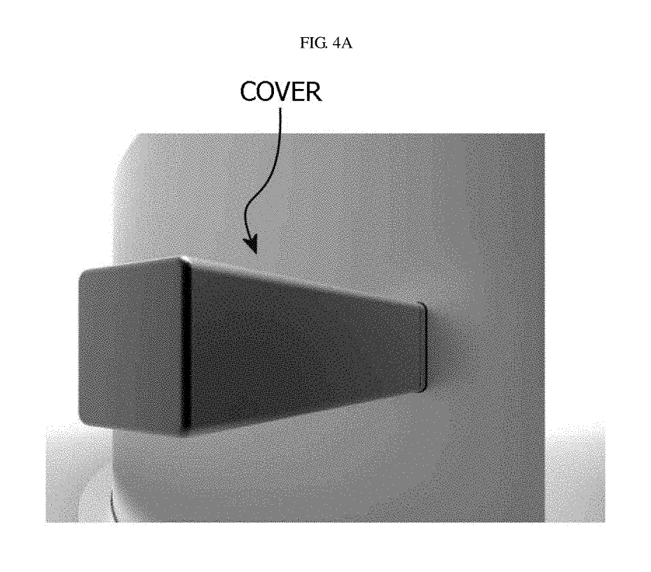

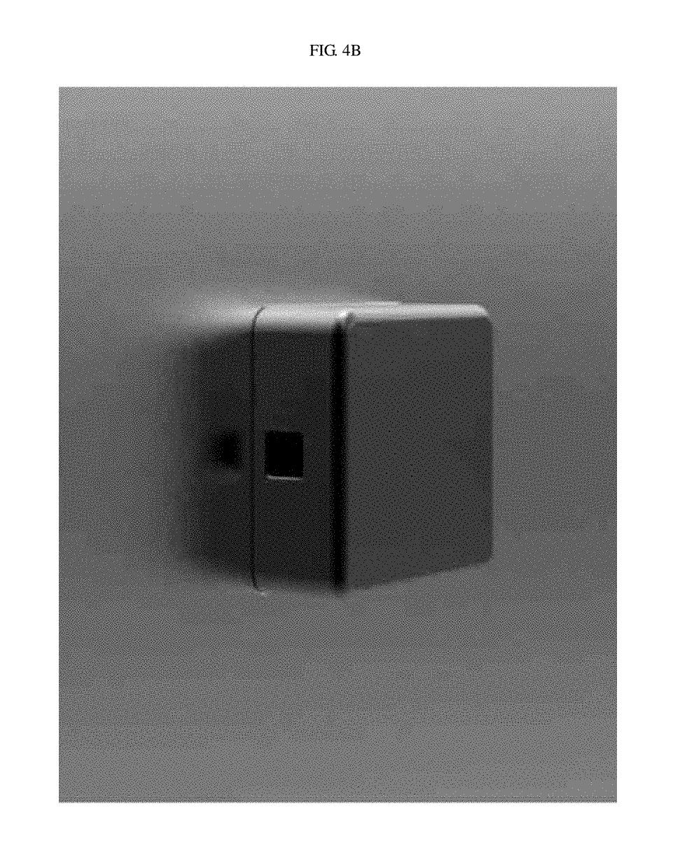

FIGS. 4A and 4B are schematic diagrams for describing a method of mounting the suspension cell culture monitoring apparatus according to an embodiment of the present invention illustrated in FIG. 1 on an incubator. FIG. 4A shows a shape of an outer portion of the suspension cell culture monitoring apparatus 100 mounted on the incubator while a cover is attached to the entire suspension cell culture monitoring apparatus 100 according to the present embodiment, and FIG. 4B shows a shape of an inner portion of the suspension cell culture monitoring apparatus 100 (that is, a shape of a portion of the suspension cell culture monitoring apparatus 100 submerged in the incubator) mounted on the incubator while the cover is attached to the suspension cell culture monitoring apparatus 100.

In this case, since the cover is formed of a waterproof material and integrally manufactured with the suspension cell culture monitoring apparatus 100, all of the elements 110 to 140 of the suspension cell culture monitoring apparatus 100, excluding the openings 111 of the chamber unit 110, are prevented from being exposed to the culture medium so that contamination is minimized.

FIGS. 5A and 5B are exemplary views illustrating a schematic configuration of a monitoring computer connected to the suspension cell culture monitoring apparatus according to an embodiment of the present invention. As illustrated in FIG. 5A, the monitoring computer 200 may be connected to one or more suspension cell culture monitoring apparatuses 100 and simultaneously monitor the one or more suspension cell culture monitoring apparatuses 100.

The monitoring computer 200 may receive an image (image of suspension cells) sent from each suspension cell culture monitoring apparatus 100 and generate statistical data. As illustrated in FIG. 5B, the suspension cell culture monitoring apparatus 100 may be synchronized in real time with at least one of an external server (e.g., a cloud server) 300, a user computer 400, and a portable wireless terminal 500 and send the image (image of suspension cells) and statistical data by using a predesignated communication method (e.g., wired, wireless, or the like).

In this case, the calculation unit 150 of the suspension cell culture monitoring apparatus 100 may perform the function of the monitoring computer 200. In this case, the calculation unit 150 may be connected to a calculation unit 150 of another suspension cell culture monitoring apparatus 100 via a communication network or may also be connected to the server 300 or the portable wireless terminal 500 via a communication network. Accordingly, the statistical data and an alarm may also be output through at least one of the server, the user computer, and the portable wireless terminal which is connected to the calculation unit 150 via a communication network.

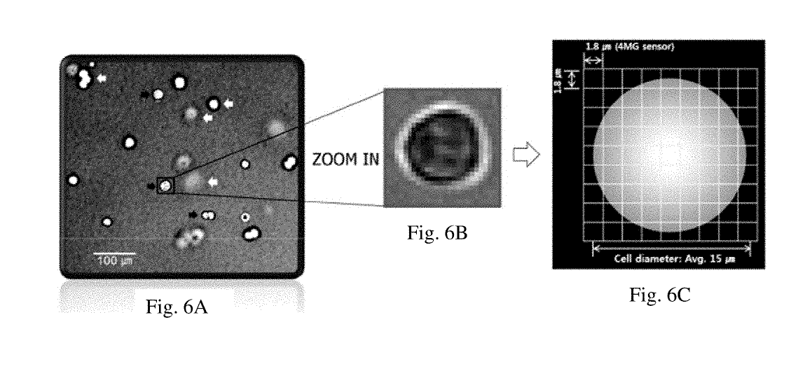

FIGS. 6A-6C are exemplary views for describing a monitoring operation of the monitoring computer illustrated in FIGS. 5A and 5B in more detail.

When an image of suspension cells is captured using the suspension cell culture monitoring apparatus 100 according to the present embodiment, as illustrated in FIG. 6A, cells which are in focus and cells which are out of focus coexist.

In FIG. 6A, a black arrow indicates a cell which is in focus, and a white arrow indicates a cell which is out of focus. FIG. 6B is a zoomed-in image of a specific cell, and FIG. 6C is an exemplary view for describing an image resolution of the monitoring computer 200. A cell with an average diameter of 15 μm is displayed as an image in 8*8 (4 MG sensor) to 1010 (25 MG sensor) pixels. In the present embodiment, the number of pixels on which an image is formed may vary according to the size of pixels of a sensor.

Accordingly, the monitoring computer 200 analyzes all of the cells which are in focus and the cells which are out of focus to calculate statistics (cell concentration), only analyzes the cells which are in focus to calculate statistics on an average shape of the cells (cell morphology), and separately stores images of the cells used in the statistics.

When fully automatic monitoring is performed in real time, for example, the monitoring computer 200 may acquire an image by capturing suspension cells at 20 fps or higher and extract necessary statistical data. When it is attempted to calculate a flow speed of the cells, the calculation may be possible through strobe imaging speed control.

In this case, in order to measure whether air inside a culture chamber is contaminated due to fungi or the like, it is necessary to remove the culture medium from inside the culture chamber and allow air inside the culture chamber to pass through the chamber unit 110. Generally, of the fungi in the air, fungi which are medium-sized or larger have a size of about 2.5 to 10 μm. A sufficient number of images, which is large enough to allow statistical analysis, is captured to measure the number of particulates in the air. For example, images are captured for about 10 seconds using a 72 fps camera, and the number of particulates is analyzed from 720 captured images. The allowed number of particulates may be set according to a level of cleanliness designated by a country. For example, a standard number of allowed particulates may be set, and when the number of particulates exceeds the standard number, an alarm may be output. The standard number of allowed particulates may conform to the standard level of cleanliness for each country.

As described above, the monitoring computer 200 outputs an alarm when the statistical data (e.g., the density of cells, number of cell entities, morphology of cells, size of cells, rotation speed of a culture medium, color of the culture medium, contamination of the culture medium, contamination of air inside a culture chamber, and the like) deviates from a preset appropriate range.

In this case, an alarm may also be output through at least one of the external server (e.g., a cloud server) 300, the user computer 401), and the portable wireless terminal 500 which is connected to the monitoring computer 200 via a communication network.

Meanwhile, monitoring is performed at preset specific time intervals, performed continuously, or performed at a specific time designated by the user, and a monitoring result may be output in real time or at a designated time.

As described above, the suspension cell culture monitoring apparatus 100 and the monitoring computer 200 linked thereto according to the present embodiment, which constitute a single system, may determine a state of cells (e.g., the morphology, concentration, flow, or the like of cells) using at least one of a real-time monitoring method and a fully automatic monitoring method, minimize the possibility of contamination by minimizing the number of times of cell sampling that a researcher performs, allow an internal state of an incubator to be determined anytime and anywhere through a portable wireless terminal, output an alarm when it is found through automatic monitoring that statistical data of the cells has deviated from an appropriate state so that the deviation is dealt with rapidly, and reduce labor costs and material costs.

According to an aspect of the present invention, a state of suspension cell culture can be automatically monitored and an analysis result thereof can be sent to a user. In this way, the concentration and morphology of cells in a culture medium, whether the cells are contaminated, a rotation speed of the culture medium, and the like can be monitored.

The present invention has been described above with reference to embodiments illustrated in the drawings, but the embodiments are merely illustrative, and one of ordinary skill in the art should understand that various modifications may be made to the embodiments, and other equivalent embodiments are possible. Therefore, the technical scope of the present invention should be defined by the claims below.

What is claimed is:

1. A suspension cell culture monitoring apparatus comprising;
a chamber unit, which is a portion submerged in an incubator in which cells are cultured, configured to be opened at least partially by an opening so that a culture medium and suspension cells are allowed to pass therethrough;
a lens unit configured to zoom in or zoom out the suspension cells moving through the opening of the chamber unit, which are visible through an observation area of the chamber unit, by adjusting a focus; and
a sensor unit configured to acquire an image in which the suspension cells visible through the lens unit are present.

2. The suspension cell culture monitoring apparatus of claim 1, wherein:
the observation area configured to allow observation of the suspension cells is formed at one side of the chamber unit; and
an illumination unit configured to illuminate the observation area is coupled and integrally formed with the chamber unit.

3. The suspension cell culture monitoring apparatus of claim 2, wherein the illumination unit is configured to illuminate the observation area from an opposite side of the observation area by using an illumination cable.

4. The suspension cell culture monitoring apparatus of claim 2, wherein the illumination unit is formed to illuminate the observation area by sharing a lens barrel with the lens unit.

5. The suspension cell culture monitoring apparatus of claim 1, wherein the suspension cell culture monitoring apparatus is formed such that an outer portion thereof, excluding the opening of the chamber unit, is partially or entirely covered by a cover.

6. The suspension cell culture monitoring apparatus of claim 5, wherein the cover is formed of a waterproof material and sterilized.

7. The suspension cell culture monitoring apparatus of claim 1, wherein a focusing operation, a zooming operation, and an iris operation of the lens unit are controlled.

8. The suspension cell culture monitoring apparatus of claim 1, further comprising a calculation unit configured to analyze the acquired image, wherein:
the calculation unit processes the image acquired by the sensor unit, analyzes a state of cell culture to extract statistical data, and outputs an alarm when the statistical data deviates from a set appropriate range; and
the statistical data and the alarm are also output through at least one of a server, a user computer, and a portable wireless terminal which is connected to the calculation unit via a communication network.
